(19) United States
(12) Patent Application Publication
(10) Pub. No.: US 2013/0035624 A1
(43) Pub. Date: Feb. 7, 2013

(54) Contoured Support Brace

(71) Applicant: Thomas Mroz, Cleveland, OH (US)

(72) Inventor: Thomas Mroz, Cleveland, OH (US)

(21) Appl. No.: 13/566,203

(22) Filed: Aug. 3, 2012

Related U.S. Application Data
(60) Provisional application No. 61/514,983, filed on Aug. 4, 2011.

(51) Int. Cl. A61F 5/055

(52) U.S. Cl. 602/18

(57) ABSTRACT

The present invention relates to an improved head and neck support brace providing increased support for the user and reduced migration of the support brace.

CROSS-REFERENCE TO OTHER APPLICATIONS

This application claims the benefit of and hereby incorporates by reference U.S. Provisional Application No. 61/514,983, entitled CONTOURED NECK SUPPORT, and filed on Aug. 4, 2011.

### BACKGROUND OF THE INVENTION

Support and stabilization of the head and neck is beneficial in a number of different circumstances. For example, head and neck injuries often require the stabilization and support of the head or neck. Stabilizing and supporting the head and neck can prevent aggravating an existing injury, or decrease recovery time. Even without a preexisting injury, head and neck stabilization and support is often desirable to make a person more comfortable. This is particularly true during prolonged periods of sitting, as is often required while traveling on a plane, train, and bus, or while riding in a car. However, current neck and head supports provide insufficient support due to their shape, material, lack of contour to human anatomy, and migration of the device (i.e. lack of stability and optimal material to help prevent it). Migration of the device relates to the problem of the device moving from the optimal or preferred position while the device is in use. As a result, these neck and head supports used for comfort while sitting provide tend to be uncomfortable to use for long periods of time and provide insufficient support. As a result, people are often uncomfortable, rest in awkward positions due to unfavorable neck and head positions, are unable to gain meaningful rest, and possibly develop neck or shoulder pain or discomfort. A support brace providing superior support and stability and preventing migration of the support during use, would thereby make the user more comfortable would be beneficial.

### SUMMARY OF THE INVENTION

Various embodiments described herein attempt to overcome the drawbacks of the conventional techniques and devices for head and neck support and stabilization. The systems, methods, and devices described herein can offer, among other advantages, a device offering increased stabilization of a user's head and neck.

The present invention is a neck and head support brace that through its material and anatomically contoured shape provides a level of comfort currently unavailable. In one example embodiment of the invention, the shape of the support brace conforms to the shoulders, the lateral sides of the neck, the chest and the chin of the user. In certain embodiments, the support brace has chin- and chest-conforming geometry, as well as support on the sides of a user head and extending to the ears. In further example embodiments, the support brace has an angled posterior portion of both sides, and the sides will both extend to the level of the ears. The support brace thereby provides increased stability and support, and prevents the support brace from moving and shifting while in use.

The present invention also includes mechanisms to increase the stability of the support. In some example embodiments, cloth extensions that are used for support and to prevent anterior migration of the support brace during use. In some additional example embodiments, a horizontal strap connecting the left and right posterior extensions of the support brace are used for support and to prevent lateral displacement.

### BRIEF DESCRIPTION OF THE DRAWINGS

Embodiments of the present invention are illustrated by way of example and not limited to the following figures:

FIG. 1 is a front view of an exemplary embodiment of a support brace including a user.

FIG. 2 is a side view of an exemplary embodiment of a support brace including a user.

FIG. 3 is a side view of an exemplary embodiment a support brace.

FIG. 3A is a side view of an exemplary embodiment a support brace with optional cloth extensions.

FIG. 4 is a front view of an exemplary embodiment of a support brace.

FIG. 5 is a top view of an exemplary embodiment of a support brace.

FIG. 6 is a bottom view of an exemplary embodiment of a support brace.

FIG. 7 is a rear view of an exemplary embodiment of a support brace.

FIG. 8 is a front view of an exemplary embodiment of a support brace.

FIG. 9 is a side view of an exemplary embodiment of a support brace.

FIG. 10 is rear view of an exemplary embodiment of a support brace.

FIG. 10A is a rear view of an exemplary embodiment of a support brace with an optional strap.

FIG. 10B is a rear view of an exemplary embodiment of a support brace with optional cloth extensions.

### DETAILED DESCRIPTION OF THE INVENTION

Reference will now be made in detail to the embodiments of the present invention, examples of which are illustrated in the accompanying drawings.

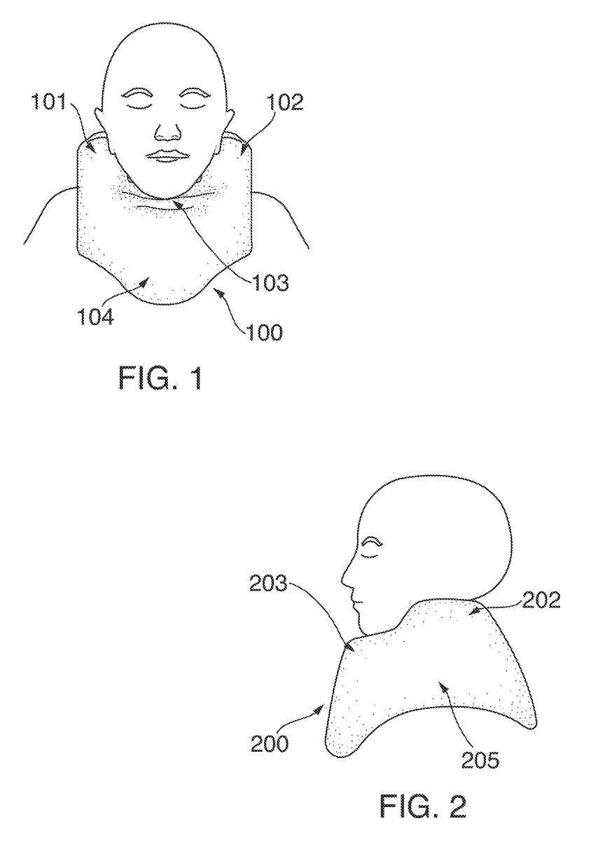

In more detail, referring to the example embodiments of the invention presented in FIGS. 1-10B, the present invention is a support brace that is contoured to human anatomy and serves to support the neck and head for the purpose of rest and comfort. In further detail, still referring to the invention of FIGS. 1-10B, the dimensions of the support brace will be adjusted based on the size of the user. For example, the support brace can be made of a variety of sizes, allowing a user to choose a size suitable for them. In other example embodiments, the support brace can be custom made for a user. The support brace may be custom made by measuring a user, or by making a mold of a user's head, neck, shoulders and chest areas. The brace is contoured to conform to the general shape of the user's body, thereby maximizing contact between the support brace and the user when used. The support brace may have differing degrees of contour to a particular user, depending on whether the support brace was specifically fit to the particular user or was selected based on the general size. Furthermore, while the support brace is described as having different portions, the support brace can be constructed as either one contiguous piece, or separate conjoining sections. In some example embodiments, custom inserts may be used in the support brace that are contoured to a specific user. These inserts maybe placed into an otherwise “off the shelf” support brace to customize the support brace for the particular user.

FIG. 1 shows a front view of an example embodiment of a support brace 100 while in use by a user. The support brace 100 includes a right portion 101, left portion 102, a chin conforming portion 103 and a chest conforming portion 104. The right portion 101 and the left portion 102 support the user's head and neck on the right and left side respectively. The right portion 101 and left portion 102 each extend to the user's ears. However, in some example embodiments, the right portion 101 and the left portion 102 may extend beyond the ears and cover all or part of the user's ears while worn. In these example embodiments, the right portion 101 and the left portion 102 may include indentations on the inside walls, allowing space for a user's ears. The right portion 101 and left portion 102 will commonly have an interior and exterior surfaces. The interior surfaces make contact with the user, while the exterior surfaces is opposite the interior surface. In some example embodiments, the interior surfaces of the right portion 101 and left portion 102 will be contoured to the shape of the side of the user's head and neck. This example embodiment would commonly include curvature to follow the user's jawline and neck.

The chin conforming portion 103 is contoured to conform to the general shape of a user's chin. In certain example embodiments, the chin conforming portion 103 includes a depression shaped similarly to the user's chin, allowing the chin to rest comfortably in the depression and also increase the surface area of contact between the user's chin and the chin conforming portion 103. In some example embodiments, the chine conforming portion 103 may be a removable and replaceable insert, thereby allowing the chin conforming portion to be easily washed or replaced as necessary.

The chest conforming portion 104 is contoured to conform to the general shape of the user's chest. As depicted in FIG. 1, the chest conforming portion 104 covers only a portion of the user's chest. In certain example embodiments, the chest conforming portion 104 may cover a larger area of the user's chest to better support the head and neck. In some example embodiments, the chest conforming portion 104 will extend approximately 6 inches down the front of the user's chest. In further example embodiments, the chest conforming portion will extent slightly below the user's collar bone. The chest conforming portion will commonly have an interior and exterior surface. The interior surface will make contact with the user, while the exterior surface is opposite the interior surface. In some example embodiments, the interior surface of the chest conforming portion 104 is contoured to the upper chest of the user.

FIG. 2 shows a side view of an example embodiment of a support brace 200 while in use by a user. The support brace includes a side portion 202, a chin conforming portion 203, and a shoulder conforming portion 205. The side portion 202 is notched and extends up to the user's ear. As depicted in FIG. 2, the side portion 202 extends up and is in contact with part of the user's head and neck, and has limited contact with the area around the users cheeks and mouth. In certain example embodiments, the size of the side portion 202 is increased to be in contact with a great portion of the user's head. The user's chin can rest on the chin conforming portion 203, which generally conforms to the shape of the user's chin. The shoulder conforming portion 205 generally conforms to the shape of a user's shoulders. As depicted in FIG. 2, the shoulder conforming portion 205 is curved and has a scalloped shape to conform to and maintain contact with the user's shoulders and chest.

In some example embodiments, the side portion 202 will extend past the user's ear for increased support and stabilization. In this example embodiment, there may be holes or depressions in the side portion 202 which would allow for the dimensions of the user's ear. In certain example embodiments, the side portions 202 may be created of a noise dampening material.

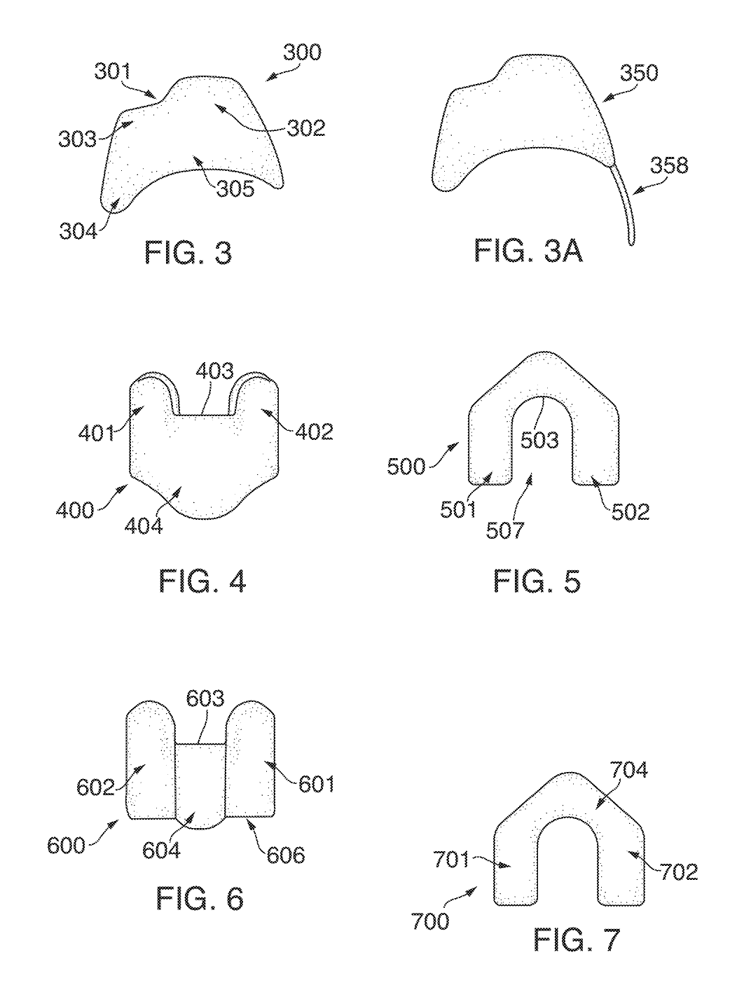

FIG. 3 shows a side view of the neck and head support brace 300. The support brace 300 includes a side portion 302, a chin conforming portion 303, a chest conforming portion 304, and a shoulder conforming portion 305. The shape of the chest conforming portion 304 generally conforms to the chest of the user, while the shoulder conforming portion 305 generally conforms to the shoulders of the user and the chin conforming portion 303 generally conforms to the chin of the user. The support brace 300 supports the user's chin anteriorly through the chin conforming portion 303 and the user's head laterally through the side portion 302. The purpose of the shape of the support brace 300 is to maximize surface area contact between the support brace 300 and the individual using the device, and thus minimize potential migration of the device and maximize the utility and comfort of it. The support brace 300 has a section notched in the side portion 302. In this manner the support brace 300 will not rub or interfere with the user's face. The side portion 302 of the support brace 300 is also angled appropriately to prevent obstruction with the back of the chair in which the user is sitting while using the support brace 300.

FIG. 3A shows the support brace 350 with an optional cloth extension 358 used in certain example embodiments to prevent anterior migration during use. The support brace 350 is similar to the support brace 300 depicted in FIG. 3 but additionally includes the optional cloth extension 358. The dimensions of the optional cloth extension 358 will be varied by size of the support brace 300 and will optimally conform to the size of the user. In some example embodiments, the cloth extension 358 will be constructed of a cloth with a rough texture and a higher coefficient of friction, to further prevent migration of the support brace 300 during use. In use, the cloth extension 358 will make contact with the user's back and prevent migration of the device through the increased friction of the device against the user. In some further example embodiments, the cloth extension 358 may include padding to offer additional comfort to the user while they are sitting.

FIG. 3A also demonstrates and optional cloth extension 358 can be an intrinsic or a removable part of the support brace 350. The purpose of the cloth extension 358, which will extend approximately 6 inches down the user's upper back, is to provide added stability to the device when the person is seated. More specifically, the cloth extension 358 will be between the user's upper back and the seat back. Therefore, when the user is seated upright, the cloth extension 358 will be held against the user's back and provide an added measure to prevent migration of the support brace 350.

FIG. 4 shows an example embodiment of a support brace 400 from a front view. The support brace 400 includes a left portion 401, a right portion 402, and a chin conforming portion 403. The support brace 400 has a chin- and chest-conforming geometry as well, as having the left portion 401 and right portion 402 of the support brace 400 extending to the ears of a user. The anterior aspect of the support brace 400 provides support of the chin through contact with the chest. The left portion 401 and right portion 402 of the support 400 are designed to reach approximately to the level of the user's ear. The chin conforming portion 403 is contoured to the user's chin and while in use allows the support brace 400 to support the weight of the user's head.

FIG. 5 shows an example embodiment of a support brace 500 from a top view. The support brace 500 has the left portion 501, right portion 502, a chin conforming portion 503, and a posterior opening 507. Support brace 500 demonstrates the “U” shaped design with the posterior aspect being non-continuous. The posterior opening 507 allows the user to slip the support brace 500 onto the user's neck by pulling the device toward the user.

FIG. 6 shows an example embodiment of the support brace 600 from the rear. The support brace 600 includes a left portion 601, a right portion 602, a chin conforming portion 603, a chest conforming portion 604, and contoured undersurface 606. Support brace 600 has an angled shape of the posterior portion of both sides, and the sides that extend to the level of the ears of the user. Support 600 has a contoured undersurface 606 as well as lateral aspects 601 and 602, which support a user's head in both the left and right direction. Contoured undersurface 606 generally conforms to the user's shoulders and allows the support brace 600 to rest on the shoulders of the user while in use. Contoured undersurface 606 can have a scalloped shape, generally in conformance with the shape of a user's shoulders. In some example embodiments, the contoured undersurface 606 will utilize a material with an increased friction, and as this portion will be in contact with the user's shoulders while in user, will thereby reduce the migration of the device while during use.

FIG. 7 shows an example embodiment of a support brace 700 from the bottom view showing chest, shoulder and neck conforming geometry. Support brace 700 includes a left portion 701, a right portion 702, a chest conforming portion 704, and a posterior opening 707. Support brace 700 has a “U” shaped design with the posterior aspect being non-continuous. Support brace 700 has a contoured underside designed to provide near anatomic matching to the user's chest and shoulders and lateral neck regions. In some example embodiments, the right portion 701 and left portion 702 have a scalloped undersurface of the anterior portion designed to provide ample space between the anterior neck and the support brace 700.

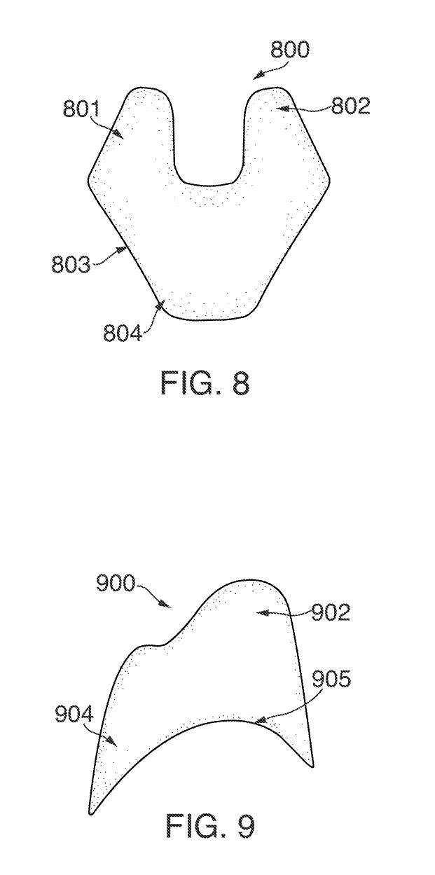

FIG. 8 shows an example embodiment of a support brace 800 from a front view showing an alternative example embodiment with a tapered shape. Support brace 800 includes a left portion 801, a right portion 802, a chin support potion 803, and a chest conforming portion 804. The right portion 801 and left portion 802 include an alternative tapered design. The width of right portion 801 and left portion 802 decreasing in the vertical direction. The chest conforming portion 804 includes an angled design that narrows from top of the chest conforming portion 804 to the bottom.

FIG. 9 shows an example embodiment of a support brace 900 from a side view showing an example embodiment with an alternative rounded shape. Support brace 900 includes a left portion 902, a chest conforming portion 904 and a shoulder conforming portion 905. The support brace 900 shows an alternative embodiment where the exterior includes a rounded geometry.

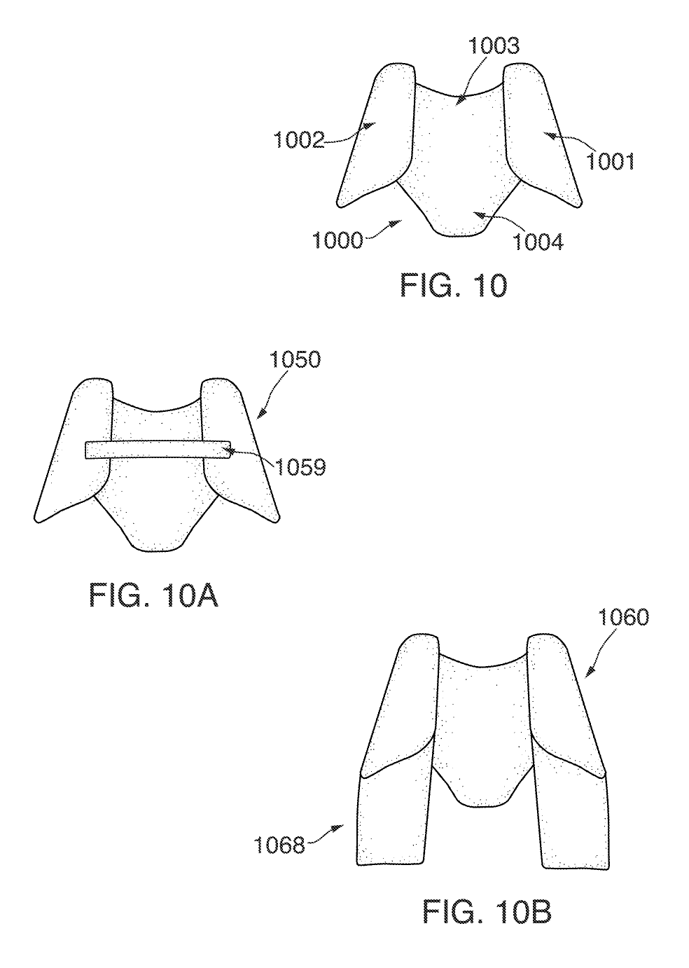

FIG. 10 shows an example embodiment of a support brace 1000 from a back view showing an alternative example embodiment with an alternative tapered shape. Support brace 1000 includes a right portion 1001, left portion 1002, chin support portion 1003 and chest conforming portion 1004. The support brace 1000 includes a tapered design where the right portion 1001 and left portion 1002 are angled on the exterior and bottom.

FIG. 10A shows support brace 1050 with an optional strap 1059 used in certain example embodiments to provide further support to the support brace 1050 and to prevent anterior migration during use and lateral displacement of the sides of the support brace 1050 during use. The support brace 1050 is similar to the brace 1000 depicted in FIG. 10 but additionally includes the strap 1059. The dimensions of the strap 1059 will be varied by size of the support brace 1050 and will optimally conform to the size of the user. The strap 1059 secures the support brace 1050 and is attached to the posterior of the support brace 1050. The strap 1059 holds the support brace in place while it is in use. In some example embodiments, the strap can include Velcro with a reciprocal Velcro section on the support brace, allowing the strap to be held in place to the posterior of the support brace 1050. In some additional example embodiments, two straps are used, with the straps connected by using any number of common fastening mechanisms. In some example embodiments, the strap 1059 will be able to be shortened or lengthened by the user, allowing the user to secure the support brace 1050 further.

FIG. 10B shows support brace 1060 with an optional cloth extensions 1068 used in certain example embodiments to provide further support to the support brace 1060 and to prevent anterior migration during use. The support brace 1060 is similar to the brace 1000 depicted in FIG. 10 but additionally includes the optional cloth extension 1068. The dimensions of the optional cloth extension 1068 will be varied by size of the support brace 1060 and will optimally conform to the size of the user. In some example embodiments, the cloth extension 1068 will be constructed of a cloth with a rough texture and a higher coefficient of friction, to further prevent migration of the support brace 1060 during use. In use, the cloth extension will make contact with the user's back and the back of the chair and prevent migration of the device through the increased friction of the device against the user. In some further example embodiments, the cloth extension 1068 may include padding to offer additional comfort to the user while they are sitting. In some example embodiments, one or more cloth extensions may be used to secure the support brace 1060. In some example embodiments, two cloth extensions may be attached to the support brace, with one attached a right portion and one attached to the left portion of the support brace 1060.

In some example embodiments, the support brace may be used by the user slipping the brace onto the user's neck and shoulders. The user is then able to sit in an upright position, typical for traveling, and rest their chin on the support brace, thereby stabilizing their head and neck.

In some example embodiments of the invention the materials used in the construction of a support brace are chosen to provide both comfort and adequate stability. The material used for the inner portion of the support brace will typically be a foam composite of the appropriate density to provide the necessary firmness, and hence, the necessary support. The medial aspect of lateral portions of the support brace that contact the ears, lateral neck and head will be composed of softer foam, such as the side portion 202 depicted in FIG. 1, may be less dense, for the comfort of the user. In some example embodiments, the support brace will have an outer shell made of fabric that may be removable and washable. In some example embodiments, the outer shell of the support brace may be a rigid non-porous material, such as plastic, allowing for increased support and easy cleaning. The inferior portion of this outer shell, the aspect that is in contact with the user's chest and shoulders, may be comprised of a different fabric with a higher coefficient of friction to further prevent migration of the support brace during use. These materials are exemplary only and any materials may be used which are known and convenient.

In some further example embodiments of the invention, the support brace may include perforations to allow airflow to reach the user. The invention may also include an integrated fan that circulates air through the perforations offer more comfort to the user.

What is claimed:

1. A support brace, comprising:
a contoured chest portion configured to extend past a collarbone of a user and to conform to an upper chest of the user;
a contoured chin portion configured to conform to a chin of the user;
a right portion and a left portion configured to conform to the lateral portions of a neck of the user;
wherein, the right portion and left portion prevent lateral movement from a head of the user and the contoured chin portion supports the head of the user.

2. The support brace of claim 1, further comprising:
a horizontal strap connecting the right portion and the left portion to prevent lateral displacement.

3. The support brace of claim 1, further comprising:
a cloth extension extending for the back of the brace, configured to extend down a back of the user.

4. The support brace of clam 1, further comprising:
an outer shell composed of fabric.

5. The support brace of claim 4, wherein the outer shell is removable.

6. The support brace of claim 5, wherein the outer shell is composed of a machine washable material.

7. The support brace of claim 1, wherein the support brace is primarily composed of foam.

8. The support brace of claim 1, wherein the right portion and the left portion are primarily composed of soft foam.

9. The support brace of claim 1, further comprising:
an internal portion composed primarily of a hard foam.

10. The support brace of clam 1, wherein the right portion and the left portion have a scalloped undersurface.

11. The support brace of claim 10, wherein the scalloped undersurface conforms to a shoulder of the user.

12. The support brace of claim 1, further comprising:
an angled back portion configured to allow a user to sit while using the brace.

13. The support brace of claim 1, wherein the support brace is a contiguous piece of foam material.
